(19) United States
(12) Patent Application Publication
(10) Pub. No.: US 2024/0131500 A1
(43) Pub. Date: Apr. 25, 2024

(54) FUNCTIONALIZED LIGNOCELLULOSE COMPOUND AND USES THEREOF

(71) Applicants: Griffin William Roberts, Springboro, OH (US); Robert K. Herrington, Topeka, KS (US); Trish Lyn Jackson, Sterling, KS (US); Alain Charles Briancon, Rockville, MD (US)

(72) Inventors: Griffin William Roberts, Springboro, OH (US); Robert K. Herrington, Topeka, KS (US); Trish Lyn Jackson, Sterling, KS (US); Alain Charles Briancon, Rockville, MD (US)

(21) Appl. No.: 18/381,837

(22) Filed: Oct. 18, 2023

Related U.S. Application Data
(60) Provisional application No. 63/417,526, filed on Oct. 19, 2022.

(51) Int. Cl. B01J 31/06; B01J 35/64; C09K 17/32; C12P 19/00

(52) U.S. Cl. CPC B01J 31/06; B01J 35/647; C09K 17/32; C12P 19/00

(57) ABSTRACT

Provided is a methodology to improve soil performance by dispersing stabilized low C:N non-highly polymerized porous lignocellulose catalytic that improves porosity, carbon capture, and microbial activities.

### BACKGROUND

### 1. Field of Invention

The present disclosure generally relates to functionalized biomass-based compounds, their manufacturing, their dispensing into lignocelluloses, their impact over time, and economic implications.

### 2. BACKGROUND OF INVENTION

Three types of plant food are applied to soils to improve their performance and quality: fertilizers, liming materials, and pesticides/herbicides.

Fertilizers are materials of natural or synthetic origin applied to soil or plant tissues to supply plant nutrients. For most modem agricultural practices, fertilization focuses on three main macronutrients: Nitrogen (N), Phosphorus (P), and Potassium (K), with the occasional addition of supplements like rock dust for micronutrients. Using large agricultural equipment or hand-tool methods, farmers apply these fertilizers using dry, pelletized, or liquid application processes.

Liming materials are calcium-rich and magnesium-rich materials in various forms, including marl, chalk, limestone, burnt lime, or hydrated lime. In acid soils, liming materials act as bases and neutralize soil acidity. This neutralization improves plant growth and increases the activity of soil bacteria. Oversupply of liming materials may result in injury to plant life.

Pesticides are poisonous substances used to kill organisms deemed harmful to the cultivation of plants or animals. They include herbicides, insecticides, nematicides, molluscicides, piscicides, avicides, rodenticides, bactericides, repellents, antimicrobials, fungicides, and lampricides.

When persistently applied, various plant foods accumulate in the soil, which may be harmful to beneficial microbes and pollinators. Most pesticides are water-soluble and can leach and enter the water table and enter potential food products. This means we need to find ways to reduce the need for and use of pesticides.

In addition to plant food, soil amendment is also added to soil to improve its physical qualities, fertility (ability to provide nutrition for plants), and mechanical characteristics. Soil amendments improve poor soils or rebuild soils damaged by improper soil management. They make poor soils more usable and maintain the soil in peak conditions.

Fertilizers are a significant source of agricultural waste because they contain an abundance of the macronutrients mentioned previously, allowing plants to grow faster and increase yields. However, when fertilizers are released into the open environment, this leads to the eutrophication of the aquatic environment. Fertilizers also significantly affect soil health, defined as the continued capacity of soil to function as a vital living ecosystem that sustains plants, animals, and humans.

Biotic factors are the living components of an ecosystem. Fertilizers impact the biotic factors of an ecosystem indiscriminately, disturbing the natural equilibrium between elements of the three classes of biotic factors: namely producers or autotrophs (i.e., plants), consumers or heterotrophs (i.e., mammals and birds), and decomposers or detritivores (i.e., microbes, insects, or worms).

Fertilizers feed plants directly and do affect the inherent physical characteristics of the soil they are applied to, a significant shortcoming.

Supply chain challenges have shown the dependency of fertilizer manufacturing on critical materials prompting a renewed interest in locally obtainable material that helps with farming.

The world produces a nearly limitless supply of biomass annually. Biomass is renewable organic material that comes from plants and animals. Plants produce biomass through photosynthesis. Biomass contains stored chemical energy from the sun. Biomass can be burned directly for heat or converted to renewable liquid and gaseous fuels through various processes. Biomass continues to be an essential fuel in many countries, especially for cooking and heating in developing countries. Biomass fuels for transportation and electricity generation are increasing in many developed countries to avoid carbon dioxide emissions from fossil fuel use.

Biomass sources for energy include:
a. Wood and wood processing wastes-firewood, wood pellets, and wood chips, lumber and furniture mill sawdust and waste, and black liquor from pulp and paper mills.
b. Biogenic materials in municipal solid wastepaper, cotton, and wool products, and food, yard, and wood wastes.
c. Human sewage (The disposal of urban sewage is a challenge.)
d. Crops and waste materials-corn, soybeans, sugar cane, switchgrass, woody plants, bacteria, algae, harvest, food processing residues, and manure. Agricultural waste is unwanted waste produced because of farming activities.

Biomass conversion has heretofore been carried out through three methods, each with limitations:
a. High-temperature carbonization/pyrolysis creating biochar
b. Hydrothermal carbonization creating hydrochar
c. Crystallization extracting phosphate, calcium phosphate, typically mixed metal, iron, zinc, or manganese

Traditionally, these methods have been utilized to produce renewable energy products via direct combustion of solid products or other upgrading strategies to produce liquid transportation fuels. These methods' solid residue (side products) has not been the subject of many studies.

Biochar is produced through the dry thermochemical process of torrefaction or pyrolysis of biomass. The different types of reactors used for biomass torrefaction are fixed bed, rotary drum, microwave, fluidized bed, and horizontal and vertical moving bed.

The high-temperature pyrolysis is akin to coffee torrefaction. The biomaterial is heated, vaporized, and condensed to create fuel (about 70%). When grass is a significant biomass component, the residue material has a high proportion of carbon (typically 60-70% for the dry matter).

Black carbon plays a vital role in the global carbon budget (at times referred to as the C budget) due to its potential to act as a significant sink of atmospheric CO₂. The similar carbon mineralization between different black carbon-rich soils regardless of soil texture underpins the importance of chemical recalcitrance for the stability of black carbon, in contrast to adjacent grounds, which showed the highest mineralization in the sandiest soil.

Biochar application to soil is a carbon-negative technology used to tackle climate change while sustainably improving soil fertility. There is a general agreement that the low degradability of biochar, like other types of black carbon, derives mainly from its specific chemistry, which is dominated by fused aromatic ring structures. Despite its low intrinsic biodegradability, the introduction of biochar to the soil often results in an increase in CO₂ emissions in the short term.

Black carbon plays a vital role in the global carbon budget because it can act as a significant sink of atmospheric CO₂. The similar C mineralization between different BC-rich soils regardless of soil texture underpins the importance of chemical recalcitrance for the stability of BC, in contrast to adjacent soils, which showed the highest mineralization in the sandiest soil.

Biochar material is suitable for carbon capture, but it does not participate in the soil, dramatically reducing its utility. There is a clear need for material that does not depend on initial soil properties to be effective and predictable.

Hydrothermal carbonization (HTC) is a wet thermochemical conversion process by which water is used as both a catalyst and a reactant for the deconstruction of commonly bio-based materials. During hydrothermal processing, water exists in a superheated liquid state at or above its saturation pressure with three general classifications based on operating temperature including carbonization (180° C.-250° C.), liquefaction (250° C.-350° C.), and gasification (>350° C.), producing primarily carbon-product forms of solid hydrochar, liquid bio-crude oil, and gas respectively. Hydrothermal processing also results in an aqueous co-product containing water-soluble/extractible carbon and ionic salts/minerals alongside a small amount of residual gasses, mainly CO₂. Hot compressed water or sub-critical water exhibits characteristics that differ significantly from ambient water in terms of density, viscosity, and dielectric constant, allowing the water to act more like an organic solvent and promote the formation of H₃O⁺. These phenomena allow biomass and biomolecular compounds to be solubilized and undergo acidic chemical reactions such as hydrolysis, depolymerization, cracking, and (at increasing temperatures and time scales) re-polymerization.

The HTC process lasts from 30 minutes to 8 hours, allowing re-polymerization of sugars and other compounds. This re-polymerization significantly impacts the biotic performance of materials created with this process because the nutrition of microbes and microbiomes must then rely on a cascading effect. First, microbes will need to break down the long polymers. These long polymers might be toxic to some microbes and stop the nutritional cycle at its inception. Some of these polymers might be Polycyclic Aromatic Hydrocarbons (PAH). Larger molecules take longer to be digested as DNA might have to be altered to accept the modules resulting in the need to generate genes. This, in turn, will slow down the metabolic behavior of microbes. A measure of performance in the diversity of the biodomes is the existence of protozoa (predators), which indicate a complex ecosystem, resulting in better nutrient cycling and improving pest resistance.

A critical measure of soil health is Soil Organic Matter (SOM). SOM serves as a reservoir of nutrients for crops, provides soil aggregation, increases nutrient exchange, retains moisture, reduces compaction, reduces surface crusting, and increases water infiltration into the soil. SOM can be estimated in the field and tested to assess mineralized nitrogen, phosphorus, and sulfur available for crop production, while dispersing amounts of carbon in the soil toward microbial biomass. Any microbe, dead or alive, will contribute to the SOM. Plants create sugar through photosynthesis, which goes into the ground and contributes to the SOM. In addition, Phospholipid Fatty Acid (PFA) contributes to the SOM. The SOM impacts the amount of surface-applied herbicides and soil pH necessary to control weeds effectively. SOM also affects the potential for herbicide carryover for future crops, the amount of fertilizer needed, and the amount of lime required to raise Ph.

The hydrology of soil is an essential measure of its health. It is characterized through two key parameters: porosity and permeability. Porosity measures the void spaces in a material, whereas permeability measures the ability of a material to transmit fluids.

Porosity and permeability are related properties of any soil. Both are related to the number, size, and connections of openings in the soil. More specifically, soil porosity measures its ability to hold a fluid. It is mathematically computed as the open space in a soil sample divided by the total soil volume (solid and area). Permeability measures the ease of flow of a fluid through porous soil. A rock may be highly absorbent, but it will have no permeability if the pores are not connected. Likewise, the soil may have a few continuous cracks which allow ease of fluid flow, but when porosity is calculated, the soil doesn't seem very porous. Clay is one of the most porous soils but is one of the least permeable. Clay usually acts as an aquitard, impeding the flow of water. Gravel and sand are porous and permeable, making them suitable aquifer materials. Gravel has the highest permeability.

Another critical area of concern is the ever-increasing presence of genetically modified organisms (GMO) in farming ecosystems. GMOs impact biodiversity and are the subject of endless lawsuits when GMO drift from area to area at times solely based on weather or water patterns.

Lignocellulosic biomass, such as agricultural and forestry residues, are attractive feedstocks for biofuel and biochemical production because of the availability of a large supply at low cost and the added benefit of environmental sustainability. Lignocellulose is composed of an intricate network of cellulose, hemicellulose, and lignin, and the composition varies according to type and species. Cellulose in lignocellulosic biomass exists in the form of fiber, microfibril, and microfibril. They can be surrounded by lignin and hemicellulose.

Example of lignocellulosic material includes and is not limited to bagasse, wheat straw, barley straw, oat straw, hay straw, corn stover, wood chips, grass, paper, sugar cane, cotton, orchard trimmings, rice husks, nut hulls, nut shells, yard waste, municipal green waste, cardboard.

Lignocellulosic biomass inherits a differential degree of recalcitrance to cellulolytic enzymes. Factors such as lignin content, cellulose crystallinity, packing structure, and hemicellulose branching determine the overall ability of biomass to be saccharified. Due to the recalcitrant nature of these complex polymers, intensive thermochemical treatments have traditionally been used to produce a substrate that can be readily hydrolyzed by commercial cellulolytic enzymes or enzyme-producing microorganisms to release sugar for fermentation. In addition, pretreatment of these biomasses have inserted in the process, including lignin removal and reduction of crystallinity of cellulose enhances biomass digestibility and can be achieved with pretreatment. Various pretreatment methods, including dilute acid, steam explosion, hot water, ammonia fiber explosion, alkaline hydrolysis, oxidative delignification, biological pretreatment, and ozonation, have been investigated and used to decrease the recalcitrance of biomass through modifying physio-chemical factors.

However, these pretreatments incur the most significant cost factors in the conversion process of lignocellulosic biomass to biofuels.

The limitations of existing fertilizers, liming materials, processed biomass, processing of lignocellulosic soil amendments, pesticides, and the need to improve soil performance and microbial behavior call for new materials and processes of manufacturing thereof.

### 3. SUMMARY

The current invention overcomes the problems and disadvantages associated with the current processing of lignocellulosic biomass. The invention creates functionalized lignocellulose catalytic particles that, when interfaced with lignocellulosic biomass, create a dually functionalized lignocellulosic compound. This compound has a structure of a primary structural carbohydrate, enabling rapid enzymatic hydrolyzation for sugar production.

One embodiment of the current invention is directed to methods of manufacturing a lignocellulosic catalyst based on the processing of biomass materials that combines concurrent thermally and kinetically induced transformation to porous particles that act as a porous substrate.

One embodiment of the current invention is directed to methods of manufacturing a lignocellulosic catalyst based on the process of biomass materials that combines thermally and kinetically induced transformations to create porous substrate particles under controlled manufacturing conditions allowing for broad tuning to many microbiomes.

One embodiment of the current invention is the application of functionalized soil amendment that leverages the broad tuning to many microbiomes.

Another aspect effect is to avoid the long exposure of materials to high temperatures, thus allowing a controlled breakage of materials.

Other embodiments and advantages of the current invention are set forth in part in the description, which follows, and in part, may be obvious from this description or may be learned from the practice of the invention.

The following is a non-exhaustive listing of some aspects of the present techniques. These and other aspects are described in the following disclosure:

### 4. BRIEF DESCRIPTION OF THE DRAWINGS

The aspects mentioned above and other aspects of the present techniques will be better understood when the present application is read given the following figures in which like numbers indicate similar or identical elements:

FIG. 1A is a logical and physical architecture diagram of a hydrothermal kinetic carbonization reactor required to manufacture high-performance lignocellulosic catalyst from lignocellulosic material.

FIG. 1B is a cross section diagram of the hydrothermal kinetic carbonization reactor from FIG. 1A, required to manufacture high-performance lignocellulosic catalyst from lignocellulosic material.

FIG. 2 is a block diagram of a lignocellulosic material transformation plant implementing a hydrothermal kinetic carbonization reactor in a mobile setting.

FIG. 3 is a logical diagram of the controlling software required for the operation of a hydrothermal kinetic carbonization reactor.

FIG. 4 represents the porous functionalized lignocellulosic catalyst resulting from the operation of a hydrothermal kinetic carbonization reactor.

FIG. 5 illustrates measurements of FLCP size across multiple samples.

FIG. 6 illustrates measurements of FLCP pore size across multiple samples.

FIG. 7A illustrates the effect of Functionalized Lignocellulose Catalytic Particles (FLCP) when applied to a representative lignocellulosic material.

FIG. 7B illustrates the effect of Functionalized Lignocellulose Catalytic Particles (FLCP) when applied to a representative lignocellulosic material.

FIG. 7C illustrates the effect of Functionalized Lignocellulose Catalytic Particles (FLCP) when applied to a representative lignocellulosic material.

FIG. 7D illustrates the effect of Functionalized Lignocellulose Catalytic Particles (FLCP) when applied to a representative lignocellulosic material.

While the present techniques are susceptible to various modifications and alternative forms, specific embodiments thereof are shown by way of example in the drawings and will herein be described in detail. The drawings may not be to scale. However, it should be understood that the drawings and detailed description thereto are not intended to limit the present techniques to the particular form disclosed. Still, to the contrary, the intention is to cover all modifications, equivalents, and alternatives falling within the spirit and scope of the present techniques as defined by the appended claims.

### 5. DETAILED DESCRIPTION

The present invention now will be described more fully hereinafter in the following detailed description of the invention, in which some, but not all embodiments of the invention are described. Indeed, this invention may be embodied in many different forms and should not be construed as limited to the embodiments set forth herein; rather, these embodiments are provided so that this disclosure will satisfy applicable legal requirements.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

To mitigate the problems described herein, the inventors had to both invent solutions and, in some cases just as importantly, recognize problems overlooked (or not yet foreseen) by others in the fields of bioscience, soil management, and manufacturing. Indeed, the inventors wish to emphasize the difficulty of recognizing those problems that are nascent and will become much more apparent in the future should trends in the industry continue as the inventors expect. Further, because multiple problems are addressed, it should be understood that some embodiments are problem-specific, and not all embodiments address every problem with traditional systems described herein or provide every benefit described herein. That said, improvements that solve various permutations of these problems are described below.

The current invention implements a novel Hydrothermal Kinetic Carbonization (HKC) to manufacture a novel class of Functionalized Lignocellulose Catalytic Particles (FLCP). Dual Functionalized Lignocellulose Compound (DFLC) are created by mixing FLCPs with other materials (including but not limited to liquids) or simply by collecting FLCPs in containers. DFLCs have a broad spectrum in their uses and performances. Microbes are a minor form of life in the soil and the basic building blocks of the soil ecosystem—increased microbe population means building the foundation of higher organisms, i.e., supports more life.

FLCPs can take multiple form factors, including solid, liquid, gel. FLCPs can be created by combining DFLC with other materials used for farming. FLCP can be included in stabilized porous soil amendment (SPSA).

DFLC is a physical input compound that stimulates a biological response from the soil at its core.

The DFLCs are a “superfood” class that builds soil organic matter (SOM) and increases total soil health. The broad-spectrum superfood effect results, partially, from DFLCs distribution of particle sizes, the molecular weight of particles, and porosity of the particles. FLCPs and DFLCs are enabled through the unique (and heretofore unexpected) capabilities of the HKC process. In one embodiment, the particles have a size between 5 micrometers and 1 millimeter. This size is bigger than the much smaller particle particles for biochar (typically between 0.05 and 2 millimeters). The increased size is key to the improved performance as it enables a higher surface roughness for the DFLC.

DFLCs improve soil performance by helping the soil loosen and crumble, increasing aeration of soil as well as soil workability, by increasing water holding capacity of soil through enhanced porosity and permeability, thus helping resist drought, increasing organic and mineral substances essential to plant growth, retaining water-soluble inorganic fertilizers in the root zones and reduces leaching.

DFLCs increase Soil Organic Matter (SOM) and Cation Exchange Capacity (CEC) in the soil. CEC measures a soil's ability to hold nutrients. It is a crucial determinant of soil fertility. Soils with high CEC can hold more cations, making them sufficient in calcium, magnesium, and other cations. In one embodiment, the HKC manufacturing performs crystallization of the DFLC, resulting in the inorganic portion of DFLCs having substantial sources of cations. In another embodiment, the DFLC source of cations includes salts, phosphate salts, ortho-phosphates, and carbonates because the KHC manufacturing crystallizes them.

DFLCs increase buffering properties of the soil and can be applied to most soils and improve the biological structure of the soil by helping to neutralize both acid and alkaline soils, which regulate the soil pH value. The rapid growth of natural soil microorganisms improves SOM performance.

DFLCs increase available plant nutrients through microbial activity increase plant root mass & above-ground biomass. DFLC holds vast diversities of organics (insoluble solids, soluble, & water extractable) that, in turn, feed diverse soil microbiome, with a significant increase in both bacteria and fungi. Increased soil fungal networks allow broader nutrient communication and transport.

Because of the smaller molecules in the material, multiple classes of microbes can digest the DFLC resulting in a higher speed of metabolism. The smaller carbon numbers in the carbon chains present in the DFLC result in fast biodegradation. This degradation processing can also be performed over a range of temperatures between −5 C and 25 C. The smallest carbon chain present in the DFLC is acetic acid in one embodiment. In one embodiment, the most miniature carbon chain current in the DFLC is propionic acid. In another embodiment, the smallest carbon chain current in the DFLC is a carboxylic acid.

In another embodiment, the DFLC includes amino acids.

In other embodiment, the DFLC includes citric, malic, lactic, and tartaric acids of certain. Amino acids naturally act as chelating agents. These agents will improve the chelation of microbes to particles.

FLCP has a bonding effect on critical elements organic and inorganic of soil. In an embodiment, the FLCP bonds with microbes. In another embodiment, the FLCP bonds with minerals.

In another embodiment, the Cation Exchange Capacity (CEC) of the DFLC impacts, when dispersed, the soil CEC, thus increasing its ability to hold onto essential nutrients and providing a buffer against soil acidification.

DFLC has philic and phobic properties, resulting in high surface energy, high molecular attraction, and a strong bonding effect.

Accelerated microbe growth and activity of diverse populations cycle nutrients between air, soil, and plants. When an active microbiome exists, nutrients cycle faster, driving nitrogen and carbon into the ground through nitrogen fixation (plant & bacteria) and photosynthesis (plants)

DFLCs improve the uptake of nitrogen and other nutrients and water by plants promote the conversion of nutrient elements (hydrogen, oxygen, nitrogen, phosphorus, potassium, sulfur, calcium, magnesium, boron, chlorine, copper, iron, manganese, molybdenum, nickel, zinc, and trace elements among others) into forms available to plants.

DFLCs enhance micronutrient uptake by increasing the chelating effect of generating ions from inorganic materials inside and around the DFLC. The DFLC makes ions more mobile and water mobile, making it more viable. Adding iron to vitamins already present allows microbes to take the DFLC materials and metabolize them for plants.

In one embodiment, the DFLC has a carbon of nitrogen ratio (C:N ratio) larger than 8. This embodiment means a reduced need for external nitrogen in the soil. Thus, any—additional nitrogen dispensed in the soil will go to the plants, not the microbes. This is important when combining DLCP with fertilizer.

In one embodiment, the DFLC has been partially crystalized during manufacturing. This results in more phosphate, calcium phosphate, iron, zinc, or manganese.

FLCP (thus DFLC) is applied through dispersion. In one embodiment, the FLCP is a stabilized porous soil amendment (SPSA).

In one embodiment, the performance of FLCPs and DFLCs may include non-compound attributes. Because of the high porosity, the surface area over which microbes can attach themselves is larger than non-porous food conditioners. That means that for the same volume of material dispersed into the soil, the active area FLCPs and thus, DFLC will be larger than traditional nonporous materials. This will increase the amount of CO2 captured in the soil as more microbiomes feed on the DFLCs.

As FLCPs have tiny pores and DFLCs include FLCPs, liquid can pour inside the DFLCs and FLCPs. FLCPs attract water through porosity, creating de-facto reservoirs that can release liquid over time. In one embodiment, FLCP is dispersed in the soil to develop drought resistance.

DFLC attracts water through porosity, creating de-facto reservoirs that can release liquid over time. In one embodiment, DFLC is dispersed in the soil to develop drought resistance.

To minimize the impact of drought, the soil needs to capture the rainwater that falls on it, store as much of that water as possible for future plant use and allow for plant roots to penetrate and proliferate. These conditions can be achieved by managing SOM increasing water storage by 30,000 gallons per acre-foot for each 1% SOM. SOM also improves the soil's ability to take in water during rainfall events, assuring that more water will be stored. The ground cover also increases the water infiltration rate while evaporating soil water. When all these factors are taken together, the severity of drought and the need for irrigation are significantly reduced.

In one embodiment, the water holding capacity increased between 5% and 25%.

In one embodiment, the water holding capacity increased between 1% and 100%.

In another embodiment, the microaggregates in the soil increase between 5% and 20% over one season. In another embodiment, the macroaggregates in the soil increase between 5% and 20% over one season. Microaggregates and macroaggregates grow from season to season.

Soil permeability or hydraulic conductivity is the rate of the flow of water and air through soil materials. Many factors affect soil permeability. Soil voids create an easy path for water movement, but other factors like hydraulic gradient, soil type, texture, and particle size distribution also affect permeability. Observing soil texture, structure, consistency, color/mottling, layering, and visible pores are the most common visual inspection of permeability.

The existence and distribution of pores on FLCP and DLCP produce a high surface area. In one embodiment, the FLCP has a surface area between 1 square meter per gram to 25 square meters per gram.

The size of the soil pores is of great importance regarding the rate of infiltration (movement of water into the soil) and the rate of percolation (movement of water through the soil). Pore size and the number of pores closely relate to soil texture and structure and influence soil permeability.

The coefficient of permeability (K) is the velocity in meters or centimeters per second of water through soils. Fine-grained soils such as clays might have values of around 10-8 meters/sec or lower, or a sand and gravel formation could be 1/10 of a millimeters/sec or higher.

Soil permeability can be estimated using empirical methods like soil survey mapping, soil texture, or particle size distribution. However, various laboratory and field test methods make it just as easy to measure these properties directly. The soil type and purpose of the test, accuracy required, and specimen type influence the selected test method.

In one embodiment, DFLC improves the soil aggregation capabilities by fostering the clumping of soil material around the DFLC.

In another embodiment, FLCP reduces lignin content.

In another embodiment, FLCP reduces fiber content.

In another embodiment, FLCP fixates N2 from the atmosphere.

In another embodiment, FLCP fixates N2 without relying on an enzymatic reaction.

In another embodiment, FLCP fixates N2 without relying on an enzymatic reaction.

In another embodiment, the FLCP is the only material introduced during the manufacturing of the DFLC.

In another embodiment, DFLC reduces lignin content.

In another embodiment, DFLC reduces fiber content.

In another embodiment, DFLC fixates N2 from the atmosphere.

In another embodiment, DFLC fixates N2 without relying on an enzymatic reaction.

In another embodiment, DFLC fixates N2 without relying on an enzymatic reaction.

The distribution of the clump (soil aggregates) dimensions is not uniform, which we can use for feeding, surface area, and a slice of “clay/soil” clump. It plays to the general use of the DFLC.

In sandy soil, these clumps aggregate to hold on to the DFLC, thus enabling the creation of canals through the soil, thus opening the soil. In one embodiment, DFLC is applied to clay material allowing the channels to be created over wide depth. In another embodiment, the DFLC is applied to high clay.

Soil Amendments include products that utilize naturally occurring hemic matter or humates formed through the chemical and biological humification of plant and animal matter over millions of years. Many recognize Humates in the sustainable agriculture movement as one of the most productive inputs available to growers. They can chemically bond with nutrients in the soil for increased absorption by the plant. In addition, they increase the permeability of plant cells which has been shown to increase nutrient uptake and decrease stress on plants throughout the growing season. The overall effect is substantially increased yields. In addition, there are Soil Biotics Soil Amendments formulated from unique combinations of beneficial microbes to provide biological improvements in the soil. In another embodiment, DFLC improves the soil biotic performance. Living organisms with fully maintained intrinsic metabolism predominately anabolic metabolic processes, e.g., livestock (domestic and zoo animals) and poultry. Anabolic and catabolic metabolic processes must be kept at least in equilibrium by appropriate feeding and care. In one embodiment, FLCP improves the biotic capabilities by adding cations to the food flow to microbes.

When applied to soil, DFLC improves many performance attributes of soil. In one embodiment, DFLC improves organic carbon through enhanced biotic activity. In another embodiment, FCACs improve water holding capacity through improved porosity through soil aggregation. In another embodiment, aggregations are aggregations of silk, sand, or clay. In another embodiment, plant roots attach to the aggregated better than dust.

Applying DFLC improves water infiltration by creating spaces between DFLC aggregates creating cracks through the structure. These improvements are the rate of water that can go through per minute at the depth where water is retained (partially or fully) at the aggregate level. Water retention is characterized by the Water Holding Capacity (WHC). For every 1% increase in SOM, the WHC of soil increases between 20,000 to 30,000 gal/ac.

In one experiment, the WHC increased 9% from season to season.

In another embodiment, the soil nutrient mobility is increased. In another embodiment, the soil nutrient mobility is equalized. In another embodiment, the introduction of DFLC improves gas exchange (nitrogen, carbon dioxide, oxygen), increasing feeding time for microbes.

In another embodiment, the application of FLCP resulted in Total Living Microbial Biomass (TLBM) levels with an average greater than 4.75 μg/g. In another embodiment, the TLBM was more significant than 4 μg/g.

In another embodiment, the application of FLCP results in higher functional group diversity among the microbial communities. This increases soil respiration. In one experiment, the average increase was 84% with a minimum increase of 7% and a maximum increase of 211%. In another embodiment, after omitting the highest and lowest extremes, the improvement in soil respiration was 83% with a range of 10 to 207% increase.

In another embodiment, the application of FLCP results in a higher fungi to bacteria ratio. In one experiment, the fungi to bacteria ratio increased 7%. In another experiment on a highly tilled field, the increase was an average of 14%. Fungi are desired and often considered indicators of good soil health.

In another embodiment, applying FLCP results in more protozoa (predators) being present. Protozoa indicate the presence of a more complex ecosystem, assisting in nutrient cycling and pest resistance. In one embodiment, the protozoa population increases are above 1600%. In another embodiment, the protozoa population increases are 1000%.

In one embodiment, applying DLFC into soil induces biological nitrogen-fixation and degradation of lignocellulosic material with high carbon:nitrogen (C:N) ratio with a range of 30:1 to 150:1.

In one embodiment, applying DLFC into soil induces rapid biological nitrogen-fixation and degradation of lignocellulosic material with high carbon:nitrogen (C:N) ratio with migration from between 30:1 and 150:1 to between 5:1 and 15:1.

In one embodiment, applying DLFC into soil induces rapid biological nitrogen-fixation and degradation of lignocellulosic material with high carbon:nitrogen (C:N) ratio with migration from between 30:1 and 150:1 to between 5:1 and 15:1 between 5 and 60 hours.

In one embodiment, the DFLC pores are mesopores. In another embodiment, the pores are micropores. In another embodiment, the pores are macropores. In another embodiment, the pores span one or more mesoporous, microporous, and macroporous scales.

In one embodiment, the FLCP is applied. In another embodiment, the FLCP is applied In another embodiment, the FLCP is applied to sandy soil. to clay soil. In another embodiment, the FLCP is applied to silty soil. In another embodiment, the FLCP is applied to peaty soil. In another embodiment, the FLCP is applied to chalky soil. In another embodiment, the FLCP is applied to loamy soil.

In one embodiment, DFLC is applied to clay material, opening it and enabling improved porosity. In one embodiment, FPAC is applied to clay material, opening it and allowing improved permeability.

In some embodiments related to manufacturing, features that may not be helpful in the processing performance may be removed (which may decrease or increase the range of parameters on manufacturing and compound). This is an example of what is referred to as feature selection. Feature selection may be helpful in terms of the required time, speed of processing, resource, adaptation to specific market needs, adaptation to regulations, soil, and perspective.

HKC leverages hydrothermal effects and uses a kinetic transformation to shear material. These two methods (kinetic and hydrothermal) are performed concurrently, creating a unique porous microbial with varied particles. HKC is a wet thermochemical conversion process by which water is used as both a catalyst and a reactant to deconstruct biobased material. During hydrothermal processing, water exists in a superheated liquid state at or above its saturation pressure with three general classifications based on operating temperature including carbonization (180° C.-250° C.), liquefaction (250° C.-350° C.), and gasification (>350° C.). By introducing kinetic energy while shearing, biomass is deconstructed at the molecular level to form a peat-like or lignite-like product referred to as kinochar (kino since the kinetic processing is critical to the product performance). Kinochar is different from hydrochar created through HTC. One key difference is the absence of long molecules. During HTC, repolymerization (at times referred as aromatization or cross-linking of intermediates) of broken sugar takes place. The HKC process is rapid and violent, preventing this repolymerization. Kinochar can thus be more porous. When applied to soil, it allows different microbes to feed simultaneously and does not require the cascading effect typical of hydrochar or biochar to be effective. Bypassing the cascading effect increase the speed at which biodomes are fed.

HKC is performed inside a specialized reaction vessel/chamber. This reaction can be part of a larger plant.

HKC processing also results in an aqueous co-product containing water-soluble/extractible carbon and ionic salts/minerals alongside a small amount of residual gasses (mainly CO₂). Hot compressed water or sub-critical water, as used in HKC, exhibits characteristics that differ significantly from ambient water in terms of density, viscosity, and dielectric constant, allowing the water to act more like an organic solvent and promoting the formation of H₃O⁺. These phenomena allow biomass and biomolecular compounds to be solubilized and undergo acidic chemical reactions such as hydrolysis, depolymerization, cracking, and (at increasing temperatures and time scales) re-polymerization.

HKC processing can include the introduction of supplementary materials to the biomass. In one embodiment, one or more mineral materials are mixed with the biomass feed in the HKC reactor. In another embodiment, one or more organic materials are mixed with the biomass feed in the HKC reactor. In yet another embodiment, 3-Hydroxy-2-naphthoic acid is mixed in with biomass before the reactor.

This invention uses process intensification concepts of HKC conversion and resulting DFLC used as a catalyst to functionalize lignocellulose material.

Those skilled in the art will appreciate the advantage of not using cascading effect for feeding biodomes.

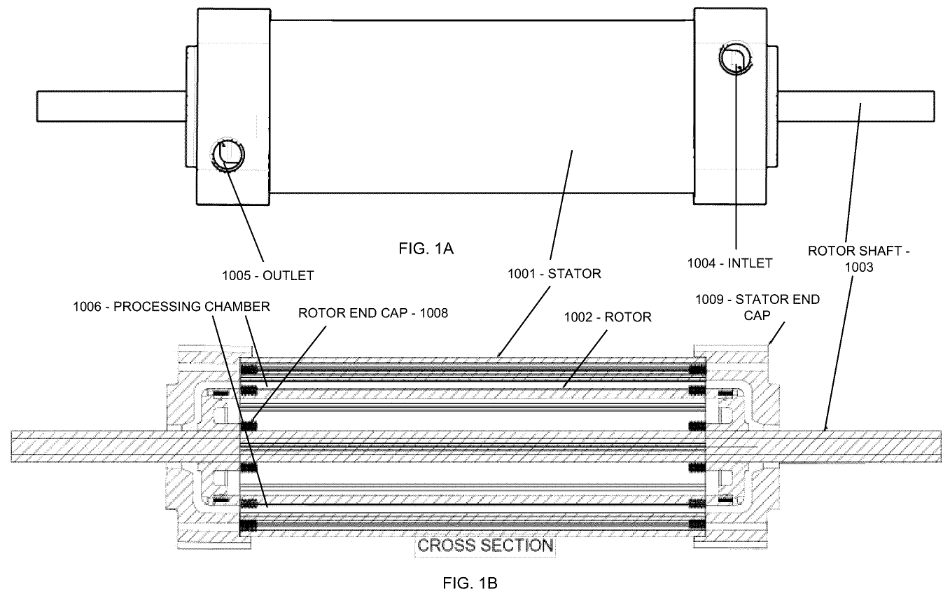

FIGS. 1A and 1B are diagrams of a hydrothermal kinetic carbonization (HKC) reactor required to manufacture high-performance functionalized porous soil amendment from biomass. The hydrothermal kinetic carbonization reactor (1000), which include a stator (1001), a rotor (1002) disposed inside the stator, a rotor shaft (1003) connected to the rotor and extending out of the stator, an inlet (1004) to allow materials (i.e., biomass, water, etc.) into the reactor, and an outlet (1005) to allow materials (i.e., biomass, water, liqueur, hydrochar, etc.) out of the reactor. FIG. 1B also illustrates on embodiment of a processing chamber (1006) which is the space defined between the rotor and the stator (1007) and is the location where the biomass is processed. The rotor can be tapered from a first end to a second end, providing a variance in the width of the processing chamber. The rotor has a rotor end cap (1008). The stator has a stator end cap (1009).

The processing chamber can have a varying width in the range of 300 to 15000 microns, 500 to 13000 microns, 500 to 12000 microns, 500 to 11000 microns, 500 to 10000 microns, 500 to 9000 microns, 500 to 8000 microns, 500 to 7000 microns, 500 to 6000 microns, 1000 to 13000 microns, 2000 to 13000 microns, 3000 to 13000 microns, 4000 to 13000 microns, 5000 to 13000 microns, 1000 to 10000 microns, 2000 to 12000 microns, 1000 to 12000 microns, 2000 to 14000 microns, 1000 to 15000 microns, 2000 to 15000 microns, 1500 to 10000 microns, or any combination thereof. The processing chamber can also have a width which is a multiple of any particle size of the unprocessed biomass selected from 0.1, 0.2, 0.3, 0.4, 0.5, 0.6, 0.7, 0.8, 0.9, 1.0, 2.0, 3.0, 4.0, 5.0, 6.0, 7.0, 8.0, 9.0 or 10.0.

Surface roughness, particularly at macro-, micro- and nano scale in fluid flow, can cause a multitude of problems in many applications. Providing materials with ultrasmooth surfaces via micro-polishing can improve performance. The rotor and the stator can each have smooth surfaces. The rotor and the stator can have a mirror or smooth surfaces. The rotor and the stator can have polished smooth surfaces. The rotor and the stator can be coated with one or more materials to provide or enhance the smoothness and/or durability of their surfaces. The coatings can be deposited in a multitude of ways including, but not limited to, electroplating, polymer coating, or a combination thereof. The coatings may be hydrophobic, oleophobic, or a combination thereof. The HKC reactor processes the unprocessed biomass in a continuous process. The HKC reactor can process biomass in the range of 98/2% solids to water or 2/98% solids to water. The HKC reactor can also process biomass in the range of 95/5%, 90/10%, 85/15%, 80/20%, 75/25%, 70/30%, 65/35%, 60/40%, 55/45%, 50/50%, 45/65%, 40/60%, 35/65%, 30/70%, 25/75%, 20/80%, 15/85%, 10/90%, or 5/95% solids to water. The HKC reactor processes biomass where it undergoes Kinetic Hydrothermal Carbonization, producing both kinochar and a liqueur. The liqueur can be subjected to further processing. The HKC reactor will operate within the hydrothermal process temperature range between 180° C. and 250° C. and at a pressure of 20-40 bar. The rotational force and kinetic energy created by the combination of the spinning rotor, the heated water, and the pressure generated within the processing chamber of the reactor help to break down the biomass through sheer force, friction, and radial force. These factors combine to break the biomass down both physically and molecularly creating kinochar.

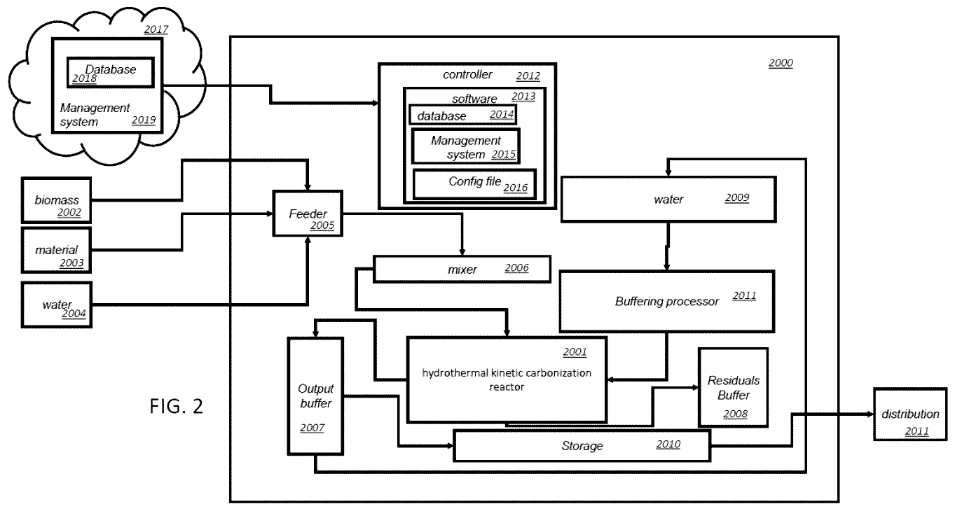

FIG. 2 is a block diagram of a biomass transformation plant (2000) implementing a hydrothermal kinetic carbonization reactor. The plant can be fixed or mobile. At the core of the plant is the hydrothermal kinetic carbonization reactor (2001). Biomass (2002), materials (2003), and water (2004) are fed into the plant through a feeder subsystem (2005). The feeder forwards these elements to a mixer (2006) that mixes these elements and then, in turn, pushes the mixture to the aforementioned hydrothermal kinetic carbonization reactor. The hydrothermal kinetic carbonization reactor produces kinochar that is delivered to an output buffering system (2007). Non-kinochar material (if any) is delivered to a residual buffer (2008). The output separates water (2009) from kinochar and feeds kinochar to storage (2010) that feeds the distribution system (2011). Some or all of the water (2009) is recycled back into the plant through a buffering processor (2011). The operation of the plant is controlled through a controller (2012) that runs software (2013), leverages a database (2014) supported by a data management system (2015). The software has configuration information (2016). The plant operation can be supported by a cloud processing (2017) that operates its own database (2018) and database management system (2019).

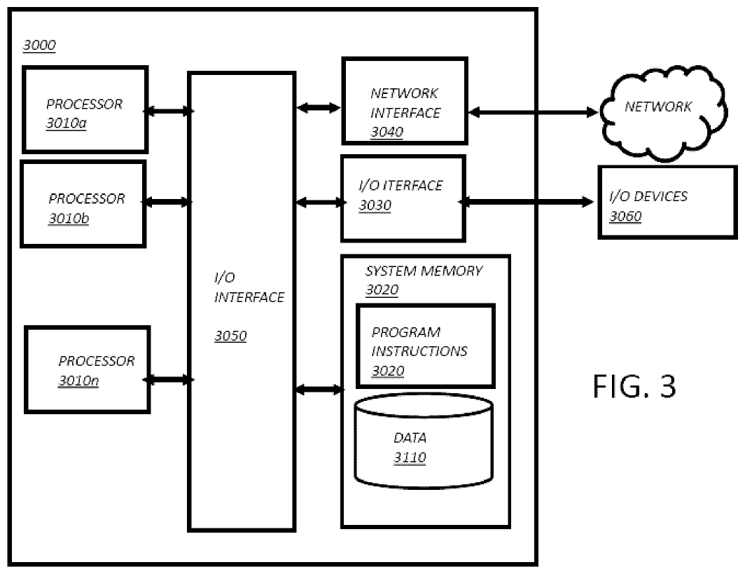

FIG. 3 is a diagram that illustrates an exemplary computing system 3000 controlling reactor operation in accordance with embodiments of the present technique. Various portions of systems and methods described herein may include or be executed on one or more computer systems similar to computing system 3000. Further, processes and modules described herein may be executed by one or more processing systems similar to that of computing system 3000.

Computing system 3000 may include one or more processors (e.g., processors 3010a-3010n) coupled to system memory 3020, an input/output I/O device interface 3030, and a network interface 3040 via an input/output (I/O) interface 3050. A processor may include a single processor or a plurality of processors (e.g., distributed processors). A processor may be any suitable processor capable of executing or otherwise performing instructions. A processor may include a central processing unit (CPU) that carries out program instructions to perform the arithmetical, logical, and input/output operations of computing system 3000. A processor may execute code (e.g., processor firmware, a protocol stack, a database management system, an operating system, or a combination thereof) that creates an execution environment for program instructions. A processor may include a programmable processor. A processor may include a Graphic Processing Unit (GPU). A processor may include general or special purpose microprocessors. A processor may receive instructions and data from a memory (e.g., system memory 3020).

Computing system 3000 may be a processor system including one processor (e.g., processor 3010a) or a multi-processor system including any number of suitable processors (e.g., 3010a-3010n). Multiple processors may be employed to provide for parallel or sequential execution of one or more portions of the techniques described herein. Processes, such as logic flows, described herein may be performed by one or more programmable processors executing one or more computer programs to perform functions by operating on input data and generating corresponding output. Processes described herein may be performed by, and apparatus can also be implemented as, special purpose logic circuitry, e.g., an FPGA (field-programmable gate array) or an ASIC (application-specific integrated circuit). Computing system 3000 may include a plurality of computing devices (e.g., distributed computer systems) to implement various processing functions.

U/O device interface 3030 may provide an interface for connection of one or more U/O devices 3060 to computer system 3000. I/O devices may include devices that receive input (e.g., from a user) or output information (e.g., to a user). I/O devices 3060 may include, for example, graphical user interface presented on displays (e.g., a cathode ray tube (CRT) or liquid crystal display (LCD) monitor), pointing devices (e.g., a computer mouse or trackball), keyboards, keypads, touchpads, scanning devices, voice recognition devices, gesture recognition devices, printers, audio speakers, microphones, cameras, or the like. U/O devices 3060 may be connected to computer system 3000 through a wired or wireless connection. I/O devices 3060 may be connected to computer system 3000 from a remote location. U/O devices 3060 located on remote computer system, for example, may be connected to computer system 3000 via a network and network interface 3040.

Network interface 3040 may include a network adapter that provides for connection of computer system 3000 to a network. Network interface 3040 may facilitate data exchange between computer system 3000 and other devices connected to the network. Network interface 3040 may support wired or wireless communication. The network may include an electronic communication network, such as the Internet, a local area network (LAN), a wide area network (WAN), a cellular communications network, or the like.

System memory 3020 may be configured to store program instructions 3100 or data 3110. Program instructions 3020 may be executable by a processor (e.g., one or more of processors 3010a-3010n) to implement one or more embodiments of the present techniques. Instructions 1300 may include modules of computer program instructions for implementing one or more techniques described herein with regard to various processing modules. Program instructions may include a computer program (which in certain forms is known as a program, software, software application, script, or code). A computer program may be written in a programming language, including compiled or interpreted languages or declarative or procedural languages. A computer program may include a unit suitable for use in a computing environment, including as a stand-alone program, a module, a component, or a subroutine. A computer program may or may not correspond to a file in a file system. A program may be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program may be deployed to be executed on one or more computer processors located locally at one site or distributed across multiple remote sites and interconnected by a communication network.

System memory 3020 may include a tangible program carrier having program instructions stored thereon. A tangible program carrier may include a non-transitory computer readable storage medium. A non-transitory computer readable storage medium may include a machine-readable storage device, a machine-readable storage substrate, a memory device, or any combination thereof. Non-transitory computer readable storage medium may include non-volatile memory (e.g., flash memory, ROM, PROM, EPROM, EEPROM memory), volatile memory (e.g., random access memory (RAM), static random-access memory (SRAM), synchronous dynamic RAM (SDRAM)), bulk storage memory (e.g., CD-ROM and/or DVD-ROM, hard drives), or the like. System memory 3020 may include a non-transitory computer readable storage medium that may have program instructions stored thereon that are executable by a computer processor (e.g., one or more of processors 3010a-3010n) to cause the subject matter and the functional operations described herein. A memory (e.g., system memory 3020) may include a single memory device and/or a plurality of memory devices (e.g., distributed memory devices). Instructions or other program code to provide the functionality described herein may be stored on a tangible, non-transitory computer-readable media. In some cases, the entire set of instructions may be stored concurrently on the media, or in some cases, different parts of the instructions may be stored on the same media at different times.

I/O interface 1050 may be configured to coordinate I/O traffic between processors 3010a-3010n, system memory 3020, network interface 3040, I/O devices 3060, and/or other peripheral devices. I/O interface 3050 may perform protocol, timing, or other data transformations to convert data signals from one component (e.g., system memory 3020) into a format suitable for use by another component (e.g., processors 3010a-3010n). I/O interface 3050 may include support for devices attached through various types of peripheral buses, such as a variant of the Peripheral Component Interconnect (PCI) bus standard or the Universal Serial Bus (USB) standard.

Embodiments of the techniques described herein may be implemented using a single instance of computer system 3000 or multiple computer systems 3000 configured to host different portions or instances of embodiments. Multiple computer systems 3000 may provide for parallel or sequential processing/execution of one or more portions of the techniques described herein.

Those skilled in the art will appreciate that computer system 3000 is merely illustrative and is not intended to limit the scope of the techniques described herein. Computer system 3000 may include any combination of devices or software that may perform or otherwise provide for the performance of the techniques described herein. For example, computer system 3000 may include or be a combination of a cloud-computing system, a data center, a server rack, a server, a virtual server, a desktop computer, a laptop computer, a tablet computer, a server device, a client device, a mobile telephone, a personal digital assistant (PDA), a mobile audio or video player, a game console, a vehicle-mounted computer, or a Global Positioning System (GPS), or the like. Computer system 3000 may also be connected to other devices that are not illustrated or may operate as a stand-alone system. In addition, the functionality provided by the illustrated components may, in some embodiments, be combined in fewer components or distributed in additional components. Similarly, in some embodiments, the functionality of some of the illustrated components may not be provided or other additional functionality may be available.

Those skilled in the art will also appreciate that while various items are illustrated as being stored in memory or on storage while being used, these items or portions of them may be transferred between memory, elements of distributed systems, and other storage devices for purposes of memory management and data integrity. Alternatively, in other embodiments, some or all of the software components may execute in memory on another device and communicate with the illustrated computer system via inter-computer communication. Some or all the system components or data structures may also be stored (e.g., as instructions or structured data) on a computer-accessible medium or a portable article to be read by an appropriate drive, various examples of which are described above. In some embodiments, instructions stored on a computer-accessible medium separate from computer system 3000 may be transmitted to computer system 3000 via transmission media or signals such as electrical, electromagnetic, or digital signals, conveyed via a communication medium such as a network or a wireless link. Various embodiments may further include receiving, sending, or storing instructions or data implemented in accordance with the foregoing description upon a computer-accessible medium. Accordingly, the present techniques may be practiced with other computer system configurations.

In block diagrams, illustrated components are depicted as discrete functional blocks, but embodiments are not limited to systems in which the functionality described herein is organized as illustrated. The functionality provided by each of the components may be provided by software or hardware modules that are differently organized than is presently depicted; for example, such software or hardware may be intermingled, conjoined, replicated, broken up, distributed (e.g., within a data center or geographically), or otherwise differently organized. The functionality described herein may be provided by one or more processors of one or more computers executing code stored on a tangible, non-transitory, machine-readable medium. In some cases, notwithstanding the use of the singular term “medium,” the instructions may be distributed on different storage devices associated with different computing devices, for instance, with each computing device having a different subset of the education, an implementation consistent with the usage of the singular term “medium” herein. In some cases, third-party content delivery networks may host some or all of the information conveyed over networks, in which case, to the extent information (e.g., content) is said to be supplied or otherwise provided, the information may be provided by sending instructions to retrieve that information from a content delivery network.

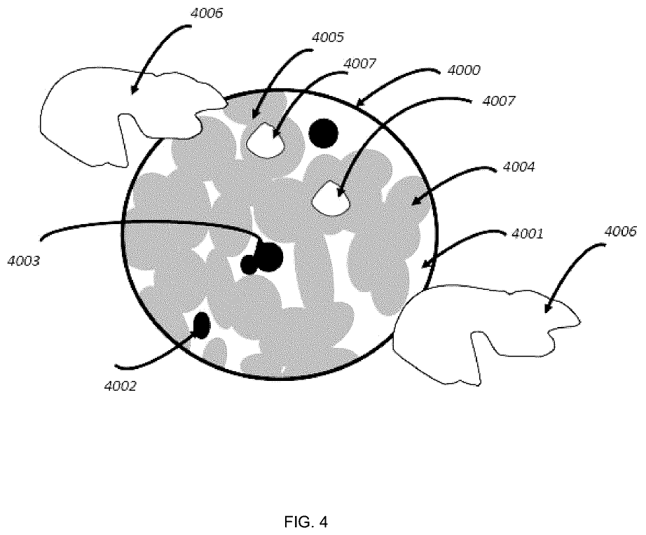

FIG. 4 shows a functional representation of a DFLC spheroid (4000) in accordance with some of the embodiments of the present techniques. A substrate (4001) which provides structure to the spheroid. The spheroid is porous with closed pores (4002), interconnected closed pores (4003), open pores (4004), and interconnected pores (4005). Organic materials (4006) are attached to the substrate. The pores hold liquids (4007).

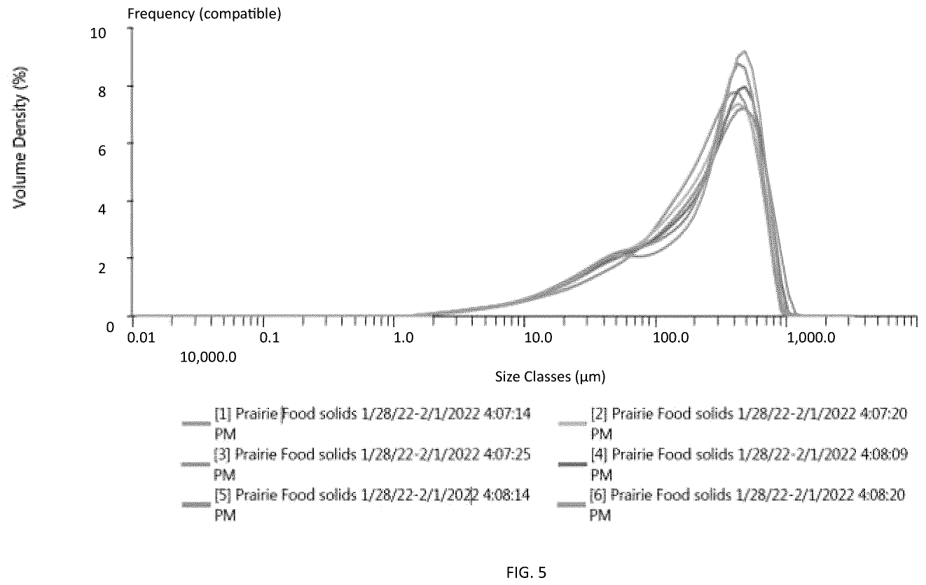

FIG. 5 illustrates the distribution of FLCP sizes that are distributed from 5 micrometers to 1 millimeter.

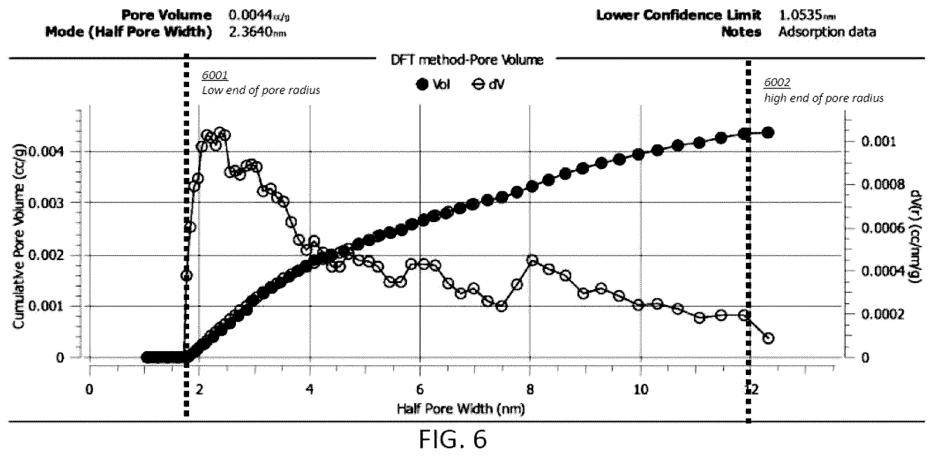

FIG. 6 illustrates the distribution of part FLCP pore sizes that are distributed from 2 nanometers in radius at the low end (6001) to 12 nanometers at the high end (6002).

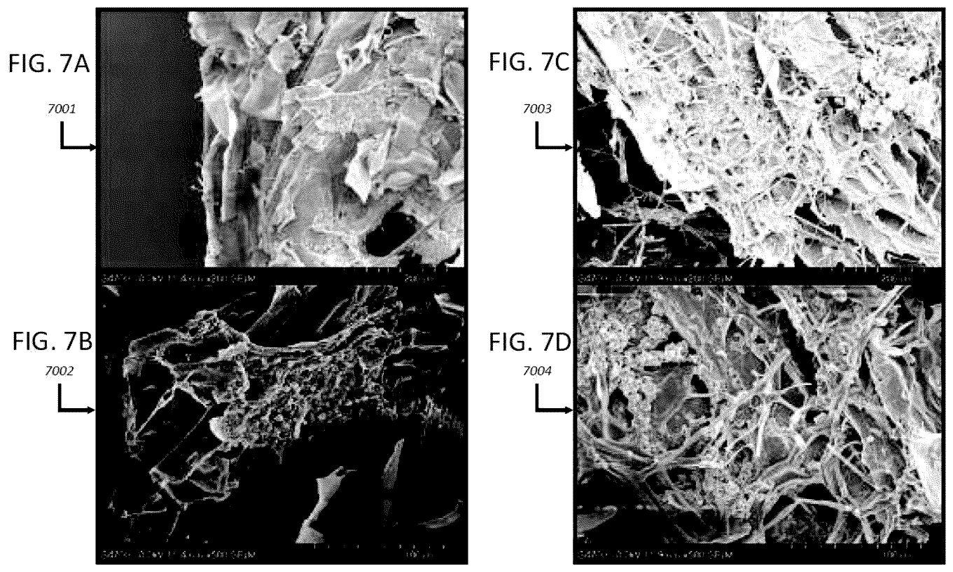

FIG. 7 illustrates the impact of adding FLPC on bagasse. Micrograph images of bagasse at 200 times magnification (7001) and 500 times magnification (7002) depict lignocellulosic structure. Micrograph images after the addition of FLPC to bagasse at 200 times magnification (7003) and 500 times magnification (7004) depict fungal response and the resultant structure.

The reader should appreciate that the present application describes several independently useful techniques. Rather than separating those techniques into multiple isolated patent applications, applicants have grouped these techniques into a single document because their related subject matter lends itself to economies in the application process. But the distinct advantages and aspects of such techniques should not be conflated. In some cases, embodiments address all of the deficiencies noted herein. Still, it should be understood that the techniques are independently helpful, and some embodiments address only a subset of such problems or offer other, unmentioned benefits that will be apparent to those of skill in the art reviewing the present disclosure. Due to costs constraints, some techniques disclosed herein may not be presently claimed and may be claimed in later filings, such as continuation applications or by amending the present claims. Similarly, due to space constraints, neither the Abstract nor the Summary of the Invention sections of the present document should be taken as containing a comprehensive listing of all such techniques or all aspects of such techniques.

It should be understood that the description and the drawings are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art given this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to) rather than the mandatory sense (i.e., meaning must). The words “include,” “including,” and “includes,” and the like mean including, but not limited to. As used throughout this application, the singular forms “a,” “an,” and “the” include plural referents unless the content explicitly indicates otherwise. Thus, for example, reference to “an element” or “an element” includes a combination of two or more elements, notwithstanding the use of other terms and phrases for one or more elements, such as “one or more.” The term “or” is, unless indicated otherwise, non-exclusive, i.e., encompassing both “and” and “or.” Terms describing conditional relationships, e.g., “in response to X, Y,” “upon X, Y,” “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed. In conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D) unless otherwise indicated. Similarly, reference to “a computer system” performing step A and “the computer system” performing step B can include the same computing device within the computer system performing both steps or different computing devices within the computer system performing steps A and B. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection has some property should not be read to exclude cases where some otherwise identical or similar members of a larger collection do not have the property, i.e., each does not necessarily mean each and every. Limitations as to the sequence of recited steps should not be read into the claims unless explicitly specified, e.g., with explicit language like “after performing X, performing Y,” in contrast to statements that might be improperly argued to imply sequence limitations, like “performing X on items, performing Y on the X'ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device. Features described with reference to geometric constructs, like “parallel,” “perpendicular/orthogonal,” “square,” “cylindrical,” and the like should be construed as encompassing items that substantially embody the properties of the geometric construct, e.g., reference to “parallel” surfaces encompasses substantially parallel surfaces. The permitted range of deviation from Platonic ideals of these geometric constructs is to be determined with reference to ranges in the specification, and where such ranges are not stated, with reference to industry norms in the field of use, and where such ranges are not defined, with reference to industry norms in the field of manufacturing of the designated feature, and where such ranges are not defined, features substantially embodying a geometric construct should be construed to include those features within 15% of the defining attributes of that geometric construct. The terms “first,” “second,” “third,” “given,” and so on, if used in the claims, are used to distinguish, or otherwise identify, and not to show a sequential or numerical limitation. As is the case in ordinary usage in the field, data structures and formats described with reference to uses salient to a human need not be presented in a human-intelligible format to constitute the described data structure or format, e.g., the text need not be rendered or even encoded in Unicode or ASCII to constitute text; images, maps, and data-visualizations need not be displayed or decoded to constitute images, maps, and data-visualizations, respectively; speech, music, and other audio need not be emitted through a speaker or decoded to constitute speech, music, or other audio, respectively. Computer-implemented instructions, commands, and the like are not limited to executable code. They can be implemented in the form of data that causes functionality to be invoked, e.g., in the form of arguments of a function or API call. To the extent bespoke noun phrases (and other coined terms) are used in the claims and lack a self-evident construction, the definition of such phrases may be recited in the claim itself, in which case, the use of such bespoke noun phrases should not be taken as an invitation to impart additional limitations by looking to the specification or extrinsic evidence.

In this patent, to the extent any U.S. patents, U.S. patent applications, or other materials (e.g., articles) have been incorporated by reference, the text of such materials is only incorporated by reference to the extent that no conflict exists between such material and the statements and drawings set forth herein. In the event of such conflict, the text of the present document governs, and terms in this document should not be given a narrower reading in virtue of the way in which those terms are used in other materials incorporated by reference.

It is noted that, as used in this specification and the appended claims, the singular forms “a,” “an,” and “the” include plural referents unless expressly and unequivocally limited to one referent.

For the purposes of this specification, unless otherwise indicated, all numbers expressing quantities of ingredients, reaction conditions, and other parameters used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed considering the number of reported significant digits and by applying ordinary rounding techniques.

All numerical ranges herein include all numerical values and ranges of all numerical values within the recited numerical ranges. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The various embodiments and examples of the present invention as presented herein are each understood to be non-limiting with respect to the scope of the invention. This is especially true with respect to the size and shape of the substrate, spheroid created, and overlap nature of the material attached to it.

Any method described herein may incorporate any design element contained within this application and any other document/application incorporated by reference herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

The present invention maybe embodied in other forms without departing from the spirit and the essential attributes thereof, and, accordingly, reference should be made to the appended claims, rather than to the foregoing specification, as indicating the scope of the invention. The invention illustratively disclosed herein suitably may be practiced in the absence of any element which is not specifically disclosed herein.

What is claimed is:

1. A functional lignocellose catalytic particle (FLCP) using for substrate a porous structure.

2. A functional lignocellulose catalytic particle such as claim 1 where said substrate is constituted of functionalized aggregates of organic and inorganic material.

3. A functional lignocellulose catalytic particle such as claim 2, where said substrate is constituted of functionalized aggregates of organic and inorganic material, is intended to catalyze a reaction or sorb a fluid component.

4. A functional lignocellulose catalytic particle such as claim 2 where said organic or inorganic materials are intended to catalyze a reaction or to sorb a fluid component.

5. A dually functionalized lignocellulose compound (DFLC) that includes a mixture of lignocellulose material and lignocellulose catalytic particles.

6. A dually functionalized lignocellulose compound such as claim 5 that includes a substrate of lignocellulose material and lignocellulose catalytic particles.

7. A dually functionalized lignocellulose compound such as claim 6 where lignocellulose material is from bagasse, wheat straw, barley straw, oat straw, hay straw, corn stover, wood chips, grass, paper, sugar cane, cotton, orchard trimmings, rice husks, nut hulls, nut shells, yard waste, municipal green waste, cardboard.

8. A method of preparing lignocellulosic biomass for enzymatic digestion, comprising:
providing lignocellulosic biomass; and
mixing said lignocellulosic biomass with functional lignocellulose catalytic particle where the ratio of FLCP mass to lignocellulosic biomass mass is between with 0.05 and 1.

9. A method of preparing lignocellulosic biomass for enzymatic digestion, comprising:
providing lignocellulosic biomass; and
mixing said lignocellulosic biomass with functional lignocellulose catalytic particle where the ratio of FLCP mass to lignocellulosic biomass mass is between with 0.01 and 2.

10. A method of inducing biological nitrogen-fixation and degradation of lignocellulosic material with high carbon:nitrogen (C:N) ratio in a range of 30:1 to 150:1.

11. A method such claim 9 where migration of degradation results in a final lignocellulosic material (C:N) ratio is between 5:1 to 15:1.

12. A method such claim 10 where migration of degradation of lignocellulosic material is between 5 and 60 hours.

13. A method such claim 10 where migration of degradation of lignocellulosic material is between 5 and 200 hours.
